(19) United States
(12) Patent Application Publication
(10) Pub. No.: US 2005/0187478 A1
(43) Pub. Date: Aug. 25, 2005

(54) Multi-wavelength imaging of highly turbid media

(75) Inventors: Pierre A. Beaudry, Pierrefonds, QC (CA); Richard Boudreault, Ville St-Laurent Quebec (CA); Julie Frechette, Sainte-Foy Quebec (CA); Sonia Verreault, St-Augustin Quebec (CA); Pierre Galarneau, Cap-Rouge Quebec (CA)

Correspondence Address: OGILVY RENAULT LLP, 1981 MCGILL COLLEGE AVENUE, SUITE 1600, MONTREAL, QC, H3A2Y3, CA

(73) Assignee: ART, Advanced Research Technologies Inc., Saint-Laurent, QC (CA)

(21) Appl. No.: 10/509,869

(22) PCT Filed: Jul. 16, 2002
(86) PCT No.: PCT/CA02/01066

Related U.S. Application Data
(60) Provisional application No. 60/305,078, filed on Jul. 16, 2001.

(51) Int. Cl. A61B 6/00

(52) U.S. Cl. 600/476

(57) ABSTRACT

In a method of multi-wavelength imaging internal structures of a highly turbid medium, the internal structures are imaged at each one of a set of at least two predetermined wavelengths, to generate a corresponding set of respective images. The set of images are then merged to generate a corresponding fused image.

### TECHNICAL FIELD

The present invention relates to imaging of turbid media, and in particular to multi-wavelength imaging of turbid media.

### BACKGROUND OF THE INVENTION

Optical imaging of turbid media typically involves launching light into the media; detecting light emerging from the media; and analyzing the detected light to infer the presence and/or properties of internal physical structures within the media. Current interest in optical imaging of turbid media stems from the need for biomedical diagnostic techniques that are safe and non-invasive. The optical properties of biological tissues are at the heart of optically based biomedical diagnostic techniques. As in the general case of any turbid medium, the manner in which light propagates through biological tissue depends on its absorption and scattering properties. In general, when the absorption and/or scattering of light traversing abnormal tissue differs from that in normal tissue (e.g. due to physiological or morphological changes resulting from the abnormality), it may be possible to optically differentiate between normal and abnormal conditions. A specific application of this concept is optical mammography, in which tumors may be differentiated from normal breast tissue on the basis of optical properties.

Biomedical optical imaging is based on the fact that the propagation of light in a turbid medium (such as biological tissue) depends on the absorption and scattering properties of the medium. Absorption results from energy level transitions of the constituent atoms and molecules in the medium. It is dependent on the material as well as the probing wavelength. Scattering results from variations in the index of refraction of the different structures present in the medium. It is dependent on the index of refraction of the structures at the probing wavelength, as well as the relative size of the structures with respect to the probing wavelength. Characteristics such as intensity, coherence and polarization of the incident light change as it is absorbed and scattered by the medium resulting in diffuse transmittance of the light. In particular, scattering causes a collimated laser beam to spread over a sizeable volume element, which complicates the imaging of a turbid medium.

The trajectory of a photon propagating inside a scattering medium can be predicted only on a statistical basis. In addition to the probability of being absorbed, the photons are subject to numerous scattering events, as shown in FIG. 1. In a slab medium that is highly scattering and weakly absorbing, such as the human breast, most photons are reflected back toward to the entrance surface after traveling only a few millimeters in the tissue. Other photons are absorbed by the medium or transmitted to the output surface where they can be detected. In the case of a typical breast thickness and optical parameters, 0.01 to 1% of incident photons are transmitted to the output surface.

The transmitted photons can be separated into three categories: ballistic photons that reach the output surface without being scattered; snake photons that are scattered slightly, but maintain an approximately rectilinear trajectory; and diffuse photons that are widely scattered and cover a considerable volume element before emerging. Exemplary trajectories followed by each of these three categories of photons are illustrated in FIG. 1.

Ballistic photons do not experience any scattering and therefore have the potential to produce a very clear image of the interior of highly turbid media such as biological tissues. Unfortunately, in many cases (e.g. for typical breast thickness and optical parameters), insufficient ballistic photons are transmitted for imaging purposes. Snake photons have an approximately rectilinear trajectory, and are sufficient in number to produce a relatively clear image. Snake photons can be differentiated from diffuse photons by their arrival time at the output surface. When a light pulse is injected into the turbid medium at the entrance surface, its component photons separate and propagate along different trajectories. The photons traveling the shortest distance (i.e. the snake photons) arrive at the output surface with the shortest propagation delay, and are thus detected before the diffuse photons, whose trajectories are longer. Thus the snake photons can be isolated by their shorter arrival time at the detector and used to construct an image. The development of this technique, known as “time gating”, caused resurgence in interest in optical mammography in the early 1990s.

As will be appreciated, time-gating involves a time-domain analysis of light received by a detector. For the purposes of biomedical imaging, such time-domain analysis and imaging is preferably based on the Temporal Point Spread Function (TPSF) of light propagating through a tissue sample (or any other turbid medium). As is known in the art, the TPSF describes the temporal divergence experienced by an ultra-short pulse of light as it propagates through a scattering medium. Thus, as shown in FIG. 1, photons of the light pulse follow different paths through the medium, and consequently experience differing propagation delays. The result is a spreading of the light pulse, in the time domain, as the pulse propagates through the medium. Evaluation of the TPSF of the pulse arriving at a detector facilitates evaluation of the absorption and scattering optical parameters of the medium, as well as attenuation. Additionally, snake-photons can be detected and used for imaging physical structures within the medium.

Typically, three parameters are defined to describe the optical properties of scattering media such as biological tissues: an absorption coefficient (μa); a scattering coefficient (μs); and an anisotropy factor (g). The absorption coefficient (μa) represents the probability of a photon being absorbed per unit of length. The scattering coefficient (μs) represents the probability of the photon being scattered per unit of length. Finally, the anisotropy factor (g) describes the average change in propagation direction associated with the scattering process.

In addition to the above three parameters, it is often useful to define a “reduced scattering coefficient” (μs′≡μs (1-g)) which represents the average distance over which a photon sustains a sufficient number of scattering events to randomize its direction of propagation. The reduced scattering coefficient (μs′) is the isotropic equivalent of the scattering coefficient (μs), and is particularly suitable in the case of thick tissue. The quantities (pa) and (μs′) are the two optical parameters generally used in highly turbid media.

In biomedical optical imaging, two types of images can be generated: 3D reconstructed images and 2D projection images. 3D reconstructed images are produced using tomography, which is typically based on a multi-point geometry involving a large number of detectors. Its advantage is that 3D images are generated. However, measurements and reconstructions are potentially time-consuming. 2D projection images are generated by scanning a small cross-section laser beam across an input surface, and detecting light emerging from a small area of the output surface as shown in FIG. 2. This scanning technique has the advantage that it is fast and compatible with time-resolved measurements (e.g. the use of time gating to detect snake photons). However, information is limited to two dimensions, the detected light giving information about a volume extending over the whole line-of-sight joining the input point of the laser beam and the detector. This is illustrated in FIG. 2, where the shaded region within the dotted lines represents the volume through which detected photons have most likely propagated. The shape of this volume can be understood by considering that all photons enter the scattering medium at the same point and all detected photons leave it through a small area facing the detector. On the other hand, scattering allows detected photons to wander away from the direct line-of-sight joining the laser source and the detector, this wandering being maximum at the half-distance between the two. Longitudinal information may be obtained in such a configuration by scanning the detector position or virtually by using method such as Dual Spatial Integration (DST) or Multiple Field-Of-View (MFOV) techniques.

As mentioned above, both scattering and absorption of photons are highly wavelength-dependent. In addition, time-gating imaging techniques require a short input optical pulse having a very sharp leading edge. As a result, conventional techniques for imaging highly turbid media utilize a laser to generate the input pulse. Such a laser generates light characterized by a very narrow range of wavelengths, all of which experience substantially identical scattering and absorption within the turbid media. A disadvantage of this arrangement is that the optical properties of most turbid media (and biological tissues in particular) are highly wavelength-dependent. Normally, the laser is tuned such that the input optical pulse will experience minimum scattering within the media, and therefore maximize the amount of light available to the detector. However, in so doing, at least some information about the internal structure of the media is lost.

Accordingly, a technique for maximizing the quality of an image of a highly turbid medium, by utilizing multiple wavelengths, remains highly desirable.

### SUMMARY OF THE INVENTION

An object of the present invention is to provide a method of multi-wavelength imaging of highly turbid media.

Thus the present invention provides a method of multi-wavelength imaging internal structures of a highly turbid medium. According to the invention, the internal structures are imaged at each one of a set of at least two predetermined wavelengths, to generate a corresponding set of respective images. The set of images are then merged to generate a corresponding fused image.

Multi-wavelength imaging in accordance with the present invention provides a tool to improve inclusion differentiation in a highly turbid medium. Specifically, several wavelengths can be used to produce a corresponding number of images of the highly turbid media, and the images subsequently combined. For optical mammography, two strategies may usefully be employed; the first strategy uses two wavelengths (e.g. 755 and 800 nm); while the second strategy uses four wavelengths (e.g. 755, 800, 930 and 975 nm). A conventional KL transform may be used to obtain the three main components of the set of images, and then pseudo-color techniques can be used to combine all the of the information in a single composite image.

### BRIEF DESCRIPTION OF THE DRAWINGS

Further features and advantages of the present invention will become apparent from the following detailed description, taken in combination with the appended drawings, in which:

FIG. 1 illustrates typical trajectories for three categories of photons transmitted through a scattering medium;

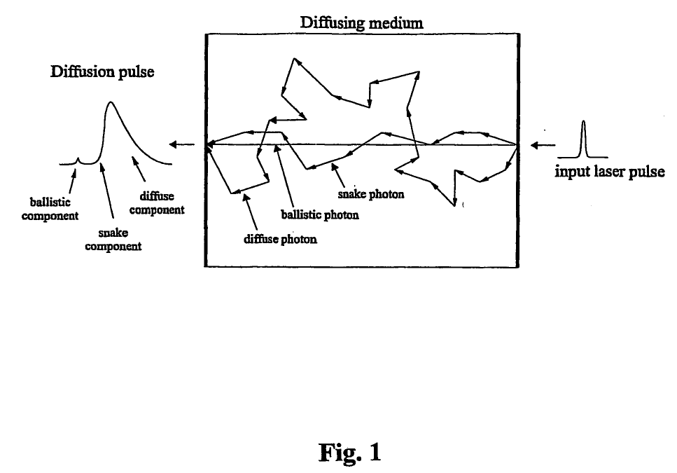

FIG. 2 illustrates a scanning system for imaging through turbid media;

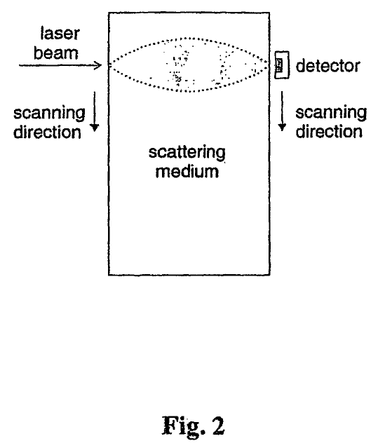

FIG. 3 illustrates the transmission spectra of adipose tissue (blue), glandular tissue (green);

FIG. 4 illustrates the transmittance (%) of cancerous (black) and glandular (green) breast tissue;

FIG. 5 illustrates the image difference technique that combines two images acquired at distinct wavelengths to enhance features that are only visible in one image;

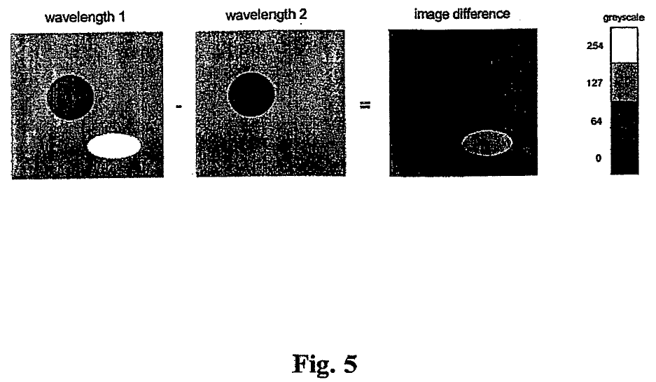

FIG. 6 illustrates the pseudo-coloring technique that combines three images into one color image where features are colored according to their intensity in the input images;

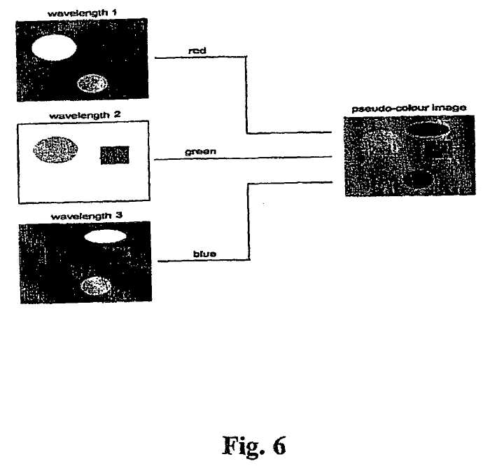

FIGS. 7A-F illustrates the image fusion results of three scans of a tissue phantom simulating different wavelengths (FIGS. 7A-C), using image difference (FIG. 7D), pseudo-coloring (FIG. 7E) and the KL transform (FIG. 7F); and

FIG. 8 illustrates the image fusion results of two in vivo scans of a human breast acquired at different wavelengths (FIGS. 8A-B), using image difference (FIG. 8C), pseudo-coloring (FIG. 8D) and the KL transform (FIG. 8E)

It will be noted that throughout the appended drawings, like features are identified by like reference numerals.

### DETAILED DESCRIPTION OF THE PREFERRED EMBODIMENT

The present invention provides a method for multi-wavelength imaging of highly turbid media. For the purposes of description, the present invention is described in detail by way of an example that is optimized for mammography. It will be appreciated, however, that the present invention can equally be applied for imaging of any turbid media. Thus it will be understood that the examples described below are in no way limitative of the scope of the present invention.

As mentioned above, the optical properties of turbid media, and in particular biological tissues, vary differently with wavelength, depending on their composition. By suitable selection of two or more wavelengths, this differing wavelength dependency can be exploited to enhance contrast between different tissue types and improve tissue recognition. By combining the images produced at each wavelength, internal features can be accentuated, resulting in improved optical contrast, tissue differentiation, and better tissue identification. As may be appreciated, the specific wavelengths used in a particular application will preferably be selected based on the optical characteristics of the turbid media under investigation, and any particular inclusions (i.e. internal structures) that are of particular interest.

For example, in optical mammography, it is desirable to distinguish the two major breast tissue types (glandular and adipose), and distinguish healthy and cancerous tissue. Glandular and adipose tissues are easily distinguished spectroscopically. In particular, glandular tissue has a large water component and its spectrum largely mimics that of water, while adipose tissue is composed largely of lipids, which are derived from fatty acids, with a smaller water component.

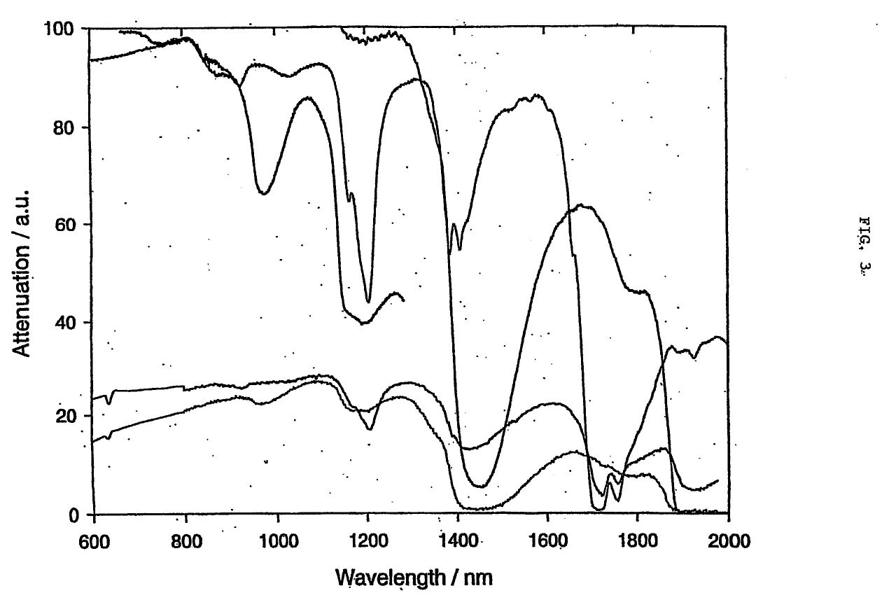

Spectra of adipose and glandular tissues are shown in FIG. 3. It should be emphasized that FIG. 3 serves only to illustrate relative peak positions with respect to wavelength. The level of attenuation cannot be used, even qualitatively, as its value for a given sample at any wavelength is dependent on sample thickness and scattering property. Two main features can be observed by examination of the tissue spectra. One is the constancy of the transmittance between 610-925 nm in the adipose tissue compared to the gradual decrease in transmittance observed over the same range in glandular tissue. Another is that a peak at 930 nm is observed in adipose tissue due to C—H bonding in lipids that is not observed in glandular tissue, whereas a water peak at 975 nm is seen with the glandular tissue but not the adipose tissue.

Thus it will be seen that imaging breast tissues at 975 nm will emphasize glandular tissues, while adipose tissues will be detected at an imaging wavelength of 930 nm. Alternatively, the invariance of the optical properties of adipose tissue between 925 nm and 610 nm could also be exploited. In this case, multi-wavelength or subtraction imaging (as described below) would be used to differentiate adipose from other tissue types.

As is known in the art, the growth of cancerous tissues are associated with a number of effects in the host tissue. One such effect is angiogenesis, which refers to new blood vessel formation and growth induced in the host tissue by release of Tumor Angiogenesis Factor (TAF) by the tumor. This hypervascularity is especially pronounced in the zone immediately surrounding the tumor, as may be seen in Table 1 below.

TABLE 1
Average red blood cell concentration (RBC) in
various tissues

| Tissue | RBC/(mm³/g) |
|---|---|
| Normal | 4.2 |
| Fibroadenoma | 5.4 |
| Carcinoma (tumor) | 4.9 |
| Carcinoma (edge) | 16.0 |
| Peripheral (tissue immediately | 9.2 |
| surrounding carcinoma) | |

This effect can be exploited in optical imaging by using an absorption peak of haemoglobin as an imaging wavelength. The isobestic point of haemoglobin, 800 nm, is the wavelength at which the absorption coefficients of oxygenated haemoglobin (HbO₂) and deoxygenated haemoglobin (Hb) and are equal, and thus gives an indication of overall haemoglobin (and thus blood) content in the tissue. Deoxygenated haemoglobin (Hb) has a weak absorption band at 755 nm. Additionally, a slightly lower degree of oxygenation has been observed (in vivo) in tumors than in healthy tissue. In fact, it is possible to define a 2-dimentional space in which HbO₂/Hb and total blood are plotted on orthogonal axes. Malignant tissues have been found to occupy a very specific area in this oxy/deoxy-total blood volume space. Accordingly, combining images produced at 755 nm and 800 nm wavelengths, respectively, can be used to exploit the effect of angiogenesis and the elevated concentration of Hb to accentuate the contrast between cancerous and normal tissue.

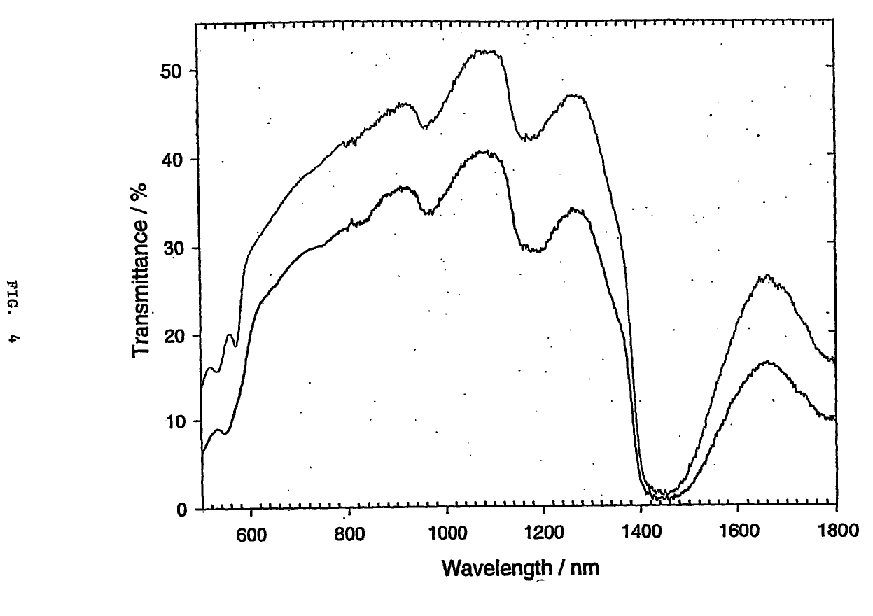

FIG. 4 shows spectra for two tissue samples with oxygenated and deoxygenated haemoglobin, respectively. As with the spectra of FIG. 3, these spectra have not been corrected for thickness and serve only to illustrate peak positioning along the wavelength axis.

It should be noted that, in all cases, it is the absorption properties that are used to differentiate tissue types. This is because the magnitude of the scattering coefficient slowly decreases with increasing wavelength, independently of tissue type, whereas the absorption coefficient is dependent on tissue composition.

In accordance with the present invention, images generated using different wavelengths are combined into a single color image that enhances the available information. This type of data processing may also be referred to as “image fusion”. Three alternative techniques of image fusion may be used, namely: image difference; pseudo-coloring; and KL transform.

Image difference refers to a technique in which one image is subtracted from another, on a pixel-by-pixel basis, to obtain a final image. Any features that are visible at only one wavelength, that is, they only appear in one image, will also be seen in the final fused image. Conversely, any features that are common to both images are subtracted out, and thus will not be seen in the fused image. The image difference technique is therefore particularly useful for identifying differences between the two images.

In practice, each image is rendered as a variable-intensity mono-chromatic (e.g. 8-bit grey-scale) image. A simple pixel-by-pixel difference calculation can then be performed, and the resultant values corrected to fit a desired range (e.g. 256 grey levels) for display on a monitor. If desired, a color map, which correlates the numbered grey levels to a given set of colors, can be used to convert the result to a color image.

A simple variation of the image-difference technique is to use image averaging, in which the intensity of each pixel of the fused image is calculated as an average of the intensities of corresponding pixels in each of the (two or more) source images, while the image differences are color coded.

The pseudo-coloring technique can be used to combine three images into a single fused color image. With this technique, each image is rendered as a variable intensity mono-chromatic image in a respective one of the three primary display colors (i.e. Red, Green and Blue). For example, the image corresponding to wavelength 1 can be rendered in variable-intensity mono-chromatic red, with each pixel being assigned an (e.g. 8-bit) intensity level. In the same manner, the images from wavelengths 2 and 3 may be rendered in mono-chromatic green and blue respectively, with their features assigned appropriate intensities. With this arrangement, the three images provide the Red, Green and Blue components of a conventional 24-bit/pixel RGB color display image. As such, the images may readily be combined, or fused, into one color image on-screen, in which features are colored according to the resultant mix of the three primary colors. For example, a feature apparent in the red and green images, but not apparent in the blue image would appear as a shade of yellow in the fused image. Note that if only two wavelengths are being considered, two primary colors are used and every pixel of the third color is set to zero (or black), so as not to interfere with the generation of the fused image.

As mentioned above, malignant tissues have been found to occupy a very specific area in an oxy/deoxy-total blood volume space. Accordingly, the pseudo-coloring technique can be used to closely identify the presence and location of malignant tissues. In particular, by generating a set of images at 755 nm (deoxygenated haemoglobin—Hb), 800 nm (total blood), each pixel of the fused image will map to a specific location in the oxy/deoxy-total blood volume space. Pixels that map into the region known to be associated with malignant tissues can then be highlighted on the display monitor.

The final image fusion technique uses the KL transform, which was originally introduced as a series expansion for continuous random processes by Karhunun and Loeve. It is also known as the method of principal components and is ideal for treating a number of images as an ensemble.

The KL transform works as follows. Assume there are N images of P pixels each, acquired at respective different wavelengths, which are written as vector components v1 . . . vN. The N×N auto-correlation matrix of the images, R, is computed by the following equation:

$$R [ i ] [ j ] = \frac{1}{P} {\sum }_{P = 1}^{P} {v}_{i} [ p ] * {v}_{j} [ p ] \qquad ( 1 )$$

where i, j ε [1,N].

The Jacobi algorithm is used to compute the N eigenvalues and eigenvectors of the auto-correlation matrix R. Then, the eigenvectors V1 . . . VN are sorted according to the corresponding eigenvalues D1 . . . DN.

The KL transformation consist of a cross-product of the sorted eigenvectors V1 . . . VN with the original images v1 . . . vN such that a new set of images k1 . . . kN of P pixels each is obtained:

$${k}_{n} [ p ] = \frac{1}{255} {\sum }_{i = 1}^{N} {V}_{n} [ i ] * {v}_{i} [ p ] \qquad ( 2 )$$

where n ε [1,N] and pε [1,p]

The N transformed images are uncorrelated. Moreover, the set of transformed images is arranged in descending order of energy. Typically, the first three images k1, k2, k3 describe over 95% of the N original images. This property makes the first three images obtained from the KL transform suitable candidates for image fusion with the pseudo-coloring technique.

The final step therefore consists of rendering each of the three images k1, k2, k3 in a respective primary color, and then combining the three colored images on-screen to produce the final fused image. If desired, the conventional LUV color system can be used (rather than RGB) to optimize the average human perception sensitivity to small color differences. Furthermore, image enhancement by histogram equalization in a manner known in the art can also be carried out.

Results from two sets of experiments are presented here, they compare the three image fusion techniques presented previously with synthetic phantoms and in a in vivo situation.

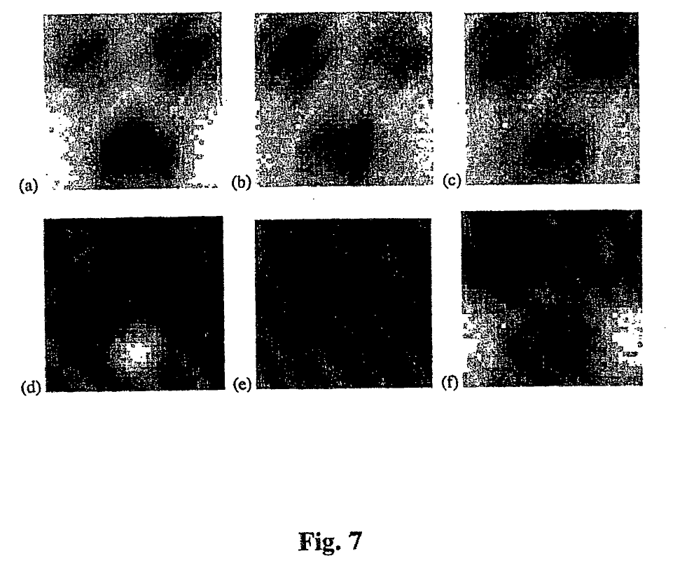

The first experiment was based on three scans of a tissue phantom containing three inclusions of different optical properties. Between each scan, the positions of the inclusions were inverted to simulate the effect of a change in wavelength. The image difference, the pseudo-coloring and the KL transform techniques were applied to the images obtained. Results of these tests, presented in FIG. 7, clearly demonstrate the improvement in inclusion differentiation and efficient data reduction technique. We notice that KL transform is particularly well adapted to multi-wavelength imaging.

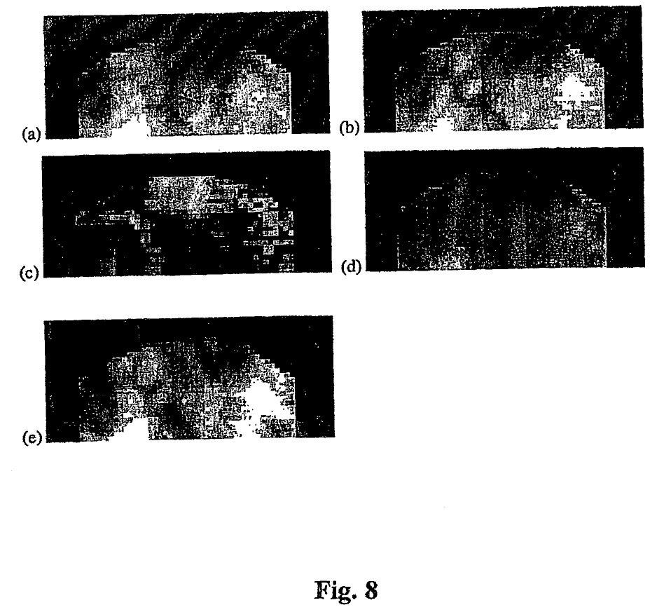

The second experiment started with the acquisition of in vivo scans from a human breast at two wavelengths (753 and 800 nm). Images were then processed using the image difference, the pseudo-coloring and the KL transform techniques. FIG. 8 presents the image fusion results obtained from the two scans. A color map has been applied to the difference images (pixel difference increases from blue to green, to yellow, then orange and finally red). As in the previous experiment, the fusion provides highlights of the image's specificity, bringing more information in a single image.

The KL transform method for image fusion, used as described previously, gives the most useful results for global visualization of multi-wavelength images. The fused color image obtained with this technique improves the contrast between the features of the input images. However, the color image obtained with the KL transform is generally less natural to the human eye that the one obtained with the pseudo-coloring technique.

As may be appreciated, the above-noted image fusion techniques may be used in combination, in order to highlight certain aspects of an image and/or as an aid to diagnostic evaluation. For example, if desired, fused images generated by the image difference and image averaging techniques may themselves be combined using the pseudo-coloring technique. In this example, a breast may be imaged at 930 nm and 975 nm, and these images combined using the image difference technique to highlight glandular and adipose tissue structures. The breast may then be imaged at 755 nm and 800 nm to determine a pixel location in the oxy/deoxy-total blood volume space. These results can then be combined into a single image by assigning, for example, Red to the average image highlighting total blood; Green to the difference image highlighting glandular and adipose tissue; and Blue to the result of mapping to the oxy/deoxy-total blood volume space, and highlighting malignant tissue.

The embodiment(s) of the invention described above is(are) intended to be exemplary only. The scope of the invention is therefore intended to be limited solely by the scope of the appended claims.

1. A method of imaging internal structures of a highly turbid medium, the method comprising steps of:
imaging the internal structures at each one of a set of at least two predetermined wavelengths, to generate a corresponding set of respective images; and
merging the set of images to generate a corresponding fused image.

2. A method as claimed in claim 1, wherein the step of imaging the internal structures is based on temporal point spread function (TPSF) analysis of light emerging from the turbid medium.

3. A method as claimed in claim 2, wherein each of the predetermined wavelengths is selected based on optical absorption properties of at least one internal structure of turbid media.

4. A method as claimed in claim 1, wherein, when the number of wavelengths is at most three, the step of merging the set of images comprises steps of:
rendering each image in a respective different primary display color of a color display monitor; and
simultaneously displaying the rendered images on the color display monitor to generate the fused image.

5. A method as claimed in claim 1, wherein, when the number of wavelengths is at most two, the step of merging the set of images comprises steps of:
rendering each image in a predetermined common primary display color of a color display monitor to generate respective rendered images; and
subtracting the rendered images to generate the fused image.

6. A method as claimed in claim 1, wherein, when the number of wavelengths is at most two, the step of merging the set of images comprises steps of:
rendering each image in a predetermined common primary display color of a color display monitor to generate respective rendered images; and
averaging the rendered images to generate the fused image.

7. A method as claimed in claim 1, wherein, when the number of wavelengths is two or more, the step of merging the set of images comprises steps of:
calculating a KL transform of each image to generate respective transformed images;
selecting at most three of the transformed images based on a respective energy of each image;
rendering each selected transformed image in a respective different primary display color of a color display monitor; and
simultaneously displaying the rendered images on the color display monitor to generate the fused image.

8. A method as claimed in claim 7, wherein the step of selecting at most three of the transformed images comprises a step of selecting transformed images having a highest energy level.

9. A method of biomedical optical imaging, the method comprising steps of:
imaging internal structures of a tissue sample at each one of a set of at least two predetermined wavelengths, to generate a corresponding set of respective images; and
merging the set of images to generate a corresponding fused image.

10. A method as claimed in claim 9, wherein the step of imaging the internal structures is based on temporal point spread function (TPSF) analysis of light emerging from the tissue sample.

11. A method as claimed in claim 9, wherein each of the predetermined wavelengths is selected based on optical absorption properties of at least one internal structure of turbid media.

12. A method as claimed in claim 11, wherein the tissue sample is breast tissue, and the set of predetermined wavelengths comprises any one or more of: 755, 800, 930 and 975 nm.

13. A method as claimed in claim 9, wherein, when the number of wavelengths is at most three, the step of merging the set of images comprises steps of:
rendering each image in a respective different primary display color of a color display monitor; and
simultaneously displaying the rendered images on the color display monitor to generate the fused image.

14. A method as claimed in claim 9, wherein, when the number of wavelengths is at most two, the step of merging the set of images comprises steps of:
rendering each image in a predetermined common primary display color of a color display monitor to generate respective rendered images;
subtracting the rendered images to generate the fused image.

15. A method as claimed in claim 9, wherein, when the number of wavelengths is at most two, the step of merging the set of images comprises steps of:
rendering each image in a predetermined common primary display color of a color display monitor to generate respective rendered images;
averaging the rendered images to generate the fused image.

16. A method as claimed in claim 9, wherein, when the number of wavelengths is two or more, the step of merging the set of images comprises steps of:
calculating a KL transform of each image to generate respective transformed images;
selecting at most three of the transformed images based on a respective energy of each image;
rendering each selected transformed image in a respective different primary display color of a color display monitor; and
simultaneously displaying the rendered images on the color display monitor to generate the fused image.

17. A method as claimed in claim 16, wherein the step of selecting at most three of the transformed images comprises a step of selecting transformed images having a highest energy level.

18. An optical imaging apparatus for imaging internal structures of a highly turbid medium, the apparatus comprising:
an optical source providing light at a plurality of wavelengths;
means for injecting said light into said medium and for recovering detection light from said medium;
means for detecting said detection light to generate raw data corresponding to said plurality of wavelengths; and
means for processing said raw data to generate an image benefiting from information gained from said plurality of wavelengths, wherein said apparatus performs the method according to any one of claims 1 to 17.
